(19) United States
(12) Patent Application Publication
(10) Pub. No.: US 2007/0041924 A1
(43) Pub. Date: Feb. 22, 2007

(54) Sebum Control Compositions Based on Saponins and Sapogenins

(75) Inventor: Shyam K Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, Scottsdale, AZ (US)

(21) Appl. No.: 11/161,856

(22) Filed: Aug. 19, 2005

(51) Int. Cl. A61K 8/63; A61K 8/60

(52) U.S. Cl. 424/70.13

(57) ABSTRACT

The present invention discloses compositions based on certain furostanol saponins and sapogenins that reduce sebum production. These compounds are useful for the reduction of excess sebum associated with dermatological disorders such as acne, dandruff, and body malodor.

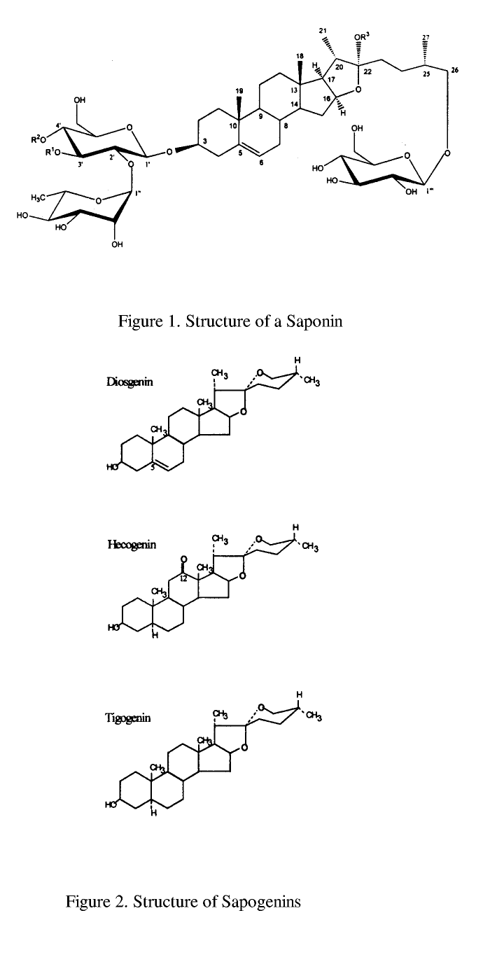

The present invention discloses compositions based on certain furostanol saponins and sapogenins that reduce sebum production. Furostanol saponins are a class of compounds that possess a furan ring fused to a steroid molecule, as shown in the partial structure in FIG. 1. Sapogenins are aglycone derivatives of such saponins, as shown in FIG. 2. These compounds, and compositions based on these compounds, are thus useful for the control of excess oil or sebum on skin. As one versed in the art can anticipate, these furostanol saponins and sapogenins can also be useful for the dermatological treatment of disorders associated with excess sebum production.

FIG. 1.

FIG. 2.

The present invention is both surprising and unexpected, especially in view of U.S. Patent application ser. no. 20030216327 (Rubinstenn et al.), which claims sapogenins to increase sebaceous secretion (sebum) for the treatment of oligoseborrheic dry skin. The present disclosure claims a reduction of sebaceous secretion, which is contrary to the above prior art. The exact biochemical mechanism of the present invention is still unknown. It is possible that Salicylic acid, Octanoyl Salicylic acid, or Retinol, which are used as additional ingredients in examples A (Lotion), B (Cream), C (Ointment), and D (Gel), F (Cream) by Rubinstenn et al. are the actual agents that provide the claimed benefits.

The oil on the surface of skin is a complex mixture of sebum, lipids (from the surface skin cells), sweat and environmental material. Sebum is produced by sebaceous glands. These are found over most of the body, although there are few on the hands or feet and none on the palms and soles. Sebaceous glands on the mid-back, forehead and chin are larger and more numerous than elsewhere (up to 400-900 glands per square centimetre). They are also numerous in the ear canal and around the genitals. The sebaceous gland consists of lobes connected by ducts, which are lined with cells similar to those on the skin surface. The sebum flow dynamics at the skin surface results from a multi-step process starting with sebocyte proliferation, intracellular lipid synthesis, cell lysis in the sebaceous duct, storage of sebum in the follicular reservoir, discharge through the follicular opening and spreading over the stratum corneum [Pierard, Dermatology, vol. 196, pages 126-129 (1998)]. Most sebaceous glands open out into the hair follicle. Some free sebaceous glands open directly onto the skin surface. These include Meibomian glands on the eyelids, Tysons glands on the foreskin and Fordyces spots on the upper lip. Sebum is produced when the sebaceous gland disintegrates. The cells take about a week from formation to discharge. Sebum is a complex and variable mixture of lipids including: Glycerides, Free fatty acids, Wax esters, Squalene, Cholesterol esters, and Cholesterol [Stewart, M. E., Semin. Dermatol. 11, 100-105 (1992)]. The action of bacterial lipases converts a varying portion of the triglycerides to free fatty acids.

The sebocyte constitutes the competent cell of the sebaceous gland. The production of sebum is associated with the program of terminal differentiation of this cell. During this differentiation, the metabolic activity of the sebocyte is essentially centered around the biosynthesis of lipids (lipogenesis), and more precisely on the neosynthesis of fatty acids and the squalene. A compound making it possible to reduce the production of the lipids constituting sebum, by the cells of the sebaceous gland (sebocytes), would therefore be of definite value for the treatment of oily skin. It will also be useful for the reduction of excess skin oil, for example, from acne. Since some of the fatty acids required for this neosynthesis may be derived from triglycerides, a lipase inhibitor such as a saponin or sapogenin could inhibit the neosynthesis of such fatty acids. However, this is just the theory at this juncture, as no prior art exists to claim the lipase inhibition by saponins and sapogenins upon their topical application. The exact mechanism of the present invention is thus unknown. This lack of scientific knowledge, however, is not to reduce the utility of the present invention in any manner.

Saponins and sapogenins have been reported to possess many useful applications in the prior art. History and uses of Dioscora plant, which contains several saponins, have been discussed by Ramberg et al. [Glycoscience and Nutrition, Vol. 3, pages 1-5 (2002)].

Diosgenin has been described as an anti-inflammatory (Yamada et al., Am. J. Physiol., 273:G355-G364, 1997); as a slimming agent, by virtue of its action on adipocytes (WO 00/30603); as a collagenase inhibitor and as an antimicrobial agent which can be used in the treatment of various pathological conditions with an infectious component, including acne and seborrheic dermatitis (DE-198 41 795). Hecogenin and tigogenin, from Agave americana, have been reported to possess anti-inflammatory benefits [Peana et al., Planta Med., 63, 199-202 (1997)]. Diosgenin has been reported to suppress 12-lipoxigenase activity [Nappez et al., Cancer Lett., vol. 4, pages 133-140 (1995)].

Cutaneous aging has been treated with various compositions comprising sapogenins, including diosgenin (U.S. Patent application Ser. No. 20020028186; U.S. Pat. No. 6,331,535; FR-2 811 561; and FR-2 811 567). Kim et al. (WO2005070436) disclose an inhibitor for the biosynthesis of gelatinase comprising ginsenoside F1 (20-O-beta-D-glucopyranosyl-20 (S)-protopanaxatriol) or compound K (20-0-beta-D-glucopyranosyl-20 (S)-protopanaxadiol), which is a chief metabolite of ginseng saponin, as an active ingredient; and a cosmetic/medical composition for the prevention of skin aging.

U.S. Patent application ser. no. 20030235599 (Besne) relates to a composition containing a sapogenin that is suitable for topical application to the skin for the smoothing out of wrinkles and fine lines. U.S. Patent application ser. no. 20030152597 (Liviero) relates to a sapogenin or of a natural extract containing it to prevent the signs of ageing of the skin, in particular the loss of elasticity and/or tonicity of the skin and/or the formation of wrinkles and fine lines, by inhibiting the activity of Collagenases. U.S. Pat. No. 6,878,367 (Picard et al.) disclose the combination of a sapogenin or a derivative or natural extract containing the same, and at least one xanthine base are useful for preventing or combating cellulite and/or for refining the figure or the contours of the face.

U.S. Patent application ser. no. 20030211185 and 2005112218 (Alexis) discloses a spirostanol saponin that is prepared from the harvested Tribulus terrestris. The enriched extract is prepared using discrete hydrolysis, separation and enrichment steps. The resulting therapeutic is useful for treating bacterial, fungal, and viral infections, particularly gynecologic infections. The antibacterial benefits of sapogenins are also claimed to treat acne (DE 198-41-795). Zhong et al. (CN1563074) disclose a steroid saponin compound, and provides its general formula. Said compound contains the straight-chain glycochain or branched chain glycochain formed from trisaccharide. Said invention also relates to preparation method of said compound and medicine composite using said compound as active component, and application of said medicine composite containing said invented compound for preparing medicine for curing superficial fungus infection and deep fungus infection.

Dioscorea tokoro extract has been described as effective for moisturizing the skin and thus softening it. Thus, document JP-10 194 947 discloses an extract of Dioscorea tokoro prepared by extraction using water. This extract, which contains a mucopolysaccharide of molecular weight of 2,000,000, is described as being of use for improving the suppleness and the moisturization of the skin. The glycoproteins, which it contains, are also thought to have a suppressive effect on sebaceous secretion. U.S. Pat. No. 5,057,502 (Walsh) similarly disclose juniper extract materials that are useful in the thinning of heavy oily, greasy secretions and giving symptom relief in human acne and other conditions of thickened secretions. The molecular structure of the biologically active molecule was shown to be an acidic polysaccharide, related to pectin (a linear polygalacturonic acid). No evidence of a saponin was shown.

Anti-obesity benefits of dioscin and diosgenin, obtained from Dioscora nipponica, have been reported [Kwon et al., Biosci. Biotechnol. Biochem., 67, 1451-1456 (2003)]. The anti-obesity benefits are claimed to originate from lipase-inhibiting activity in the digestive system of rodents, thus inhibiting triglyceride absorption. The lipase inhibition by saponins and sapogenins on topical application has not been reported. Anti-hypercholesteramic activity of sapogenins is also described by Ma et al. [Zhongguo Zhong Yao Za Zhi, vol. 27, pages 528-531 (2002)]. The saponins from garlic act as modifiers of cardiovascular disease due to their cholesterol lowering effect [Matsuura, Journal of Nutrition, vol. 131, pages 100S-1005S (2001)]. U.S. Patent application ser. no. 20030119428 (Davis et al.) provides a treatment of obesity using sterol or 5.alpha.-stanol absorption inhibitors. U.S. Pat. No. 6,150,336 (Deninno et al.) discloses steroidal glycoside derivatives useful as hypocholesteramic agents and anti-atherosclerosis agents. However, as Deninno et al. point out, pure sapogenins do not significantly inhibit cholesterol's absorption. It is only when compounded with another moiety that sapogenins have the desired effect. Examples of such sapogenin compounds are compounds of tigogenin and diosgenin, particularly glycosides thereof. U.S. Pat. Nos. 4,602,003 and 4,602,005 disclose certain steroidal glycosides, in particular 3-O-(.beta.-D- glucopyranosyl)-tigogenin and 3-O-(.beta.-D-cellobiosyl)-tigogenin and their use for the control of hypercholesterolemia. 3-O-(.beta.-D-Cellobiosyl)-tigogenin has superior hypocholesteramic activity when compared to, for example, cholestyramine. PCT publication WO 93/07167 discloses several steroidal glycosides in particular 3-O-(5-C-hydroxymethyl-L-arabino-hexopyranosyl)-tigogenin and 3-O-(5-C-hydroxymethyl-L-arabino-hexopyranosyl)-diosgenin and their use in the control of hypercholesterolemia.

The anti-proliferative and apoptosis (cancer treatment) benefits of several sapogenins have been reported by Corbier et al. [International Journal of Oncology, vol. 2, pages 899-905 (2003)]. Aculeoside B, a spirostanol saponin from Ruscus aculeatus, inhibits the growth of leukemia HL-60 cells [Mimaki et al., J. Nat. Prod., vol. 61, pages 1279-1282 (1998)]. Vijay et al. (WO2005063790) disclose a novel saponin tigogenin penta glycoside isolated from the aerial parts of Chlorophytum nimonii and a process for the isolation thereof as well as its use in anti-hyperglycemic and hypolipidemic activities.

Neurodegenerative disorders, cognitive dysfunction, non-cognitive neurodegeneration, non-cognitive neuromuscular degeneration, and receptor loss in the absence of cognitive, neural and neuromuscular impairment have been treated with steroidal sapogenins (U.S. Patent application ser. nos. 20050130948, Rees et al.; 20040147495, Barraclough at al., 20040081709, Xia et al.). Alzheimer's disease has been claimed to be treated with saponins (U.S. Pat. No. 6,812,213; Xia et al.).

U.S. Pat. No. 6,905,714 (Ong et al.) discloses a preparation of Eucommia ulmoides prepared by ethanol extraction that is useful for modulating a steroid-mediated physiological condition, wherein the steroid-mediated physiological condition is mediated by an androgen or by androgen receptor, for example, male sexual development, secondary sexual development, anabolic processes, male sex drive, skin condition, hair growth, physical stamina or lipid metabolism. The effect of skin condition modulation, for example effect on sebum production by topical application of these extracts in the absence of a steroid-mediated physiological condition was not disclosed by Ong et al.

Saponins have been reported to possess detergency properties, as claimed by Sprague et al. (U.S. patent application ser. No. 2005090565). Saponins can thus remove surface oil and sebum, for example on skin by their detergency action. However, their effect on reducing the sebum and oil biosynthesis in skin was not reported by Sprague at al.

The above prior art clearly shows that none of the furostanol saponins and sapogenins have been claimed in the prior art that reduce sebum and oil production upon their topical application on humans.

In fact, Rubisntenn et al. (JP2003300862) specifically claim a cosmetic composition containing a sapogenin selected from diosgenin and hecogenin or a sapogenin-containing plant extract that is used as a treatment agent for the hyposeborrheic dry skin or dry scalp. The sapogenin or the sapogenin-containing plant extract can also be used for the dermatological treatment method for diseases related to the hyposeborrheic dry skin. These can be applied to the treatment of skin dry out, especially to the skin of females after climacteric. Thus, Diosgenin, Hecogenin, or a sapogenin-containing plant extract promote the formation of sebum, not reduce sebum, to treat hyposeborrheic dry skin or dry scalp

The saponins and sapogenins of the present invention can be obtained from various plant sources, such as Ruscus aculeatus, Garlic, Dioscora, Tribulus terrestris, Alfalfa, Chlorophytum nimonii, and Agave. Among these, mention may be made of the Dioscorea composita, Dioscorea deltoides, Dioscorea floribunda, Dioscorea sylvatica, Dioscorea spiculiflora and Dioscorea villosa species. The specific examples include: Dioscin, Diosgenin, Hecogenin, Tigogenin, Gitogenin, Chlorogenin, Eruboside, Protoeruboside, Manogenin, Shlorogenin, Hainangenin, Protodioscin, Protodiosgenin, Aculeoside, Smilagenin, Sarsapogenin, Yamogenin, Yuccagenin, Sativoside, and their various derivatives and structural analogs. Although saponins and sapogenins both provide benefits of sebum reduction, their solubility pattern can be advantageous in formulating skin beneficial compositions. In general, saponins are more water-soluble than sapogenins, hence saponin provide better bioavailability from oil-in-water or water-in-oil emulsion-based compositions and delivery systems.

The saponins and sapogenins of the present invention can also be obtained and used in an extract form. The expression “plant extracts” is intended to mean any plant extract containing one or more of these saponins and/or sapogenins. Diosgenin can be extracted from the tubers of certain Dioscorea using a method comprising successively: hydrolysis, under hot conditions, of the heterosides in inorganic acid medium (optionally after fermentation and drying of the tubers); and filtration of the insoluble fraction, which is then neutralized, washed and treated with an apolar solvent. Hecogenin can be extracted from the leaves of Agave Sisalana. Such extracts can be further refined and obtained in a higher chemical purity by usual extraction and purification methods, for example, U.S. Pat. No. 3,981,867 (Beauvoir). Other extraction methods can also be used, for example CO2 extraction.

The amount of sapogenin, which can be used according to the present invention, depends of course on the desired effect and can therefore vary within a large range, this amount being within the skill of the ordinary artisan in view of this disclosure. To give an order of magnitude, the saponin or sapogenin can be used in an amount representing from 0.0001% to 5.0% of the total weight of a composition, preferably in an amount representing from 0.01% to 2.0% of the total weight of the composition. The plant extract containing saponin or sapogenin, can be used in an amount representing from 0.0001% to 20% of the total weight of a composition, depending on the % solids content of the plant extract and the amount and nature of the extraction solvent present in such extract.

It has additionally been discovered by the present inventor that the inclusion of zinc salts of certain hydroxy acids in combination with furostanol saponins and sapogenins of the present invention synergistically increase the sebum and skin oil reducing benefits of such furostanol saponins and sapogenins. The examples of such zinc salts of hydroxy acids include zinc Hydroxycitrate, zinc Hydroxybenzoate, zinc salicylate, zinc mandelate, zinc lactate, zinc glycolate, zinc malate, zinc tetronate, zinc tartrate, and combinations thereof. This is highly unexpected and surprising since zinc salts, such as zinc Salicylate, have been reported to possess antibacterial benefits (U.S. Patent application ser. no. 20050019288, Lemoine; U.S. Pat. No. 6,846,846, Modak et al; U.S. Pat. No. 6,656,456; Dodd et al.); and anti-irritant benefits (U.S. Pat. No. 5,985,918; Modak et al.). The sebum reducing benefits of such zinc salts, either alone, or in synergistic combinations with a furostanol saponin or sapogenin, have not been disclosed in the prior art.

Synergistic benefits are also noted when a Citrate Lyase enzyme inhibitor agent is included in combination with furostanol saponins and sapogenins of the present invention. The examples of such agents include Forskohlin, Coleus forskohlii extract, Momordica Charantia extract, Charantins, Momordicosides, Hydroxycitric acid, Garcinia cambogia extract, Phaseolamin, Phaseolus vulgaris extract, Synephrine, Hordenine, Octopamine, Tyramine, n-Methyltyramine, and combinations thereof. The sebum reducing benefits of such Citrate Lyase enzyme inhibitors, either alone, or in synergistic combinations with a furostanol saponin or sapogenin, have not been disclosed in the prior art.

For topical application to the skin, the sebum reducing agents of the present invention may be provided in any cosmetic or pharmaceutical form normally used in the cosmetics and dermatological fields, and it may in particular be in the form of an aqueous, optionally gelled, solution, of a dispersion of the optionally two-phase lotion type, of an emulsion obtained by dispersion of a fatty phase (oil) in an aqueous phase (O/W) or vice versa (W/O), of a triple emulsion (W/O/W or O/W/O) or of a vesicular dispersion of the ionic and/or nonionic type. These compositions may be prepared according to the usual methods. This composition may be more or less fluid and have the appearance of a cream, an ointment, a milk, a lotion, a serum, a paste, and a mousse. It may optionally be applied in the form of an aerosol. It may also be provided in solid form, in particular in the form of a stick. It may be used as a care product and/or as a make-up product for the skin. It may also be used as a shampoo or a conditioner.

Sebum reducing agents of the present invention can be formulated in various cosmetic and pharmaceutical consumer products utilizing a variety of delivery systems and carrier bases. Such consumer product forms include the group consisting of shampoos, aftershaves, sunscreens, body and hand lotions, skin creams, liquid soaps, bar soaps, bath oil bars, shaving creams, conditioners, permanent waves, hair relaxers, hair bleaches, hair detangling lotion, styling gel, styling glazes, spray foams, styling creams, styling waxes, styling lotions, mousses, spray gels, pomades, shower gels, bubble baths, hair coloring preparations, conditioners, hair lighteners, coloring and non-coloring hair rinses, hair grooming aids, hair tonics, spritzes, styling waxes, band-aids, and balms.

In another preferred aspect, the delivery system can be traditional water and oil emulsions, suspensions, colloids, micro emulsions, clear solutions, suspensions of nanoparticles, emulsions of nanoparticles, or anhydrous compositions.

The compositions that contain the compound of the present invention may also contain adjuvants which are used in the cosmetics field, such as hydrophilic or lipophilic gelling agents, hydrophilic or lipophilic active agents, preserving agents, antioxidants, solvents, fragrances, fillers, screening agents, pigments, odor absorbers and dyestuffs. The amounts of these various adjuvants may be those conventionally used in the field considered. These adjuvants, depending on their nature, can be introduced into the fatty phase, into the aqueous phase or into the lipid vesicles. In addition, moisturizers may complete the effect obtained using the sapogenins according to the invention and anti-inflammatory agents are also useful.

The application of sebum reducing agents of the present invention can be in several areas of consumer interest, some of which include control of excess facial oil associated with acne, control of excess oil on scalp associated with dandruff, and control of excess body and underarm oil associated with body and underarm malodor.

Dandruff is the result of the normal growing process of the skin cells of the scalp. Shedding of dead skin cells from the scalp at an excessive rate is the result of the normal growing process of the skin cells of the scalp. In a normal scalp, the process of sloughing off old cells and manufacturing of their replacements is very orderly and complete. In the dandruff scalp, there is mass disorder and often the departing cells are not dead before leaving the scalp. Contrary to popular theory, although bacteria may aggravate a dandruff condition, bacteria do not cause the initial problem. Most medical authorities consider dandruff, even the mildest forms, to be a type of scalp or skin related disease. Clinically, one description of dandruff is Seborrhea Capitos or excessive sebum production of the scalp. Today most skin specialists agree that dandruff is associated with a tiny fungus called Pityrosporum ovale, or P. ovale for short. This fungus lives on our bodies and scalp all the time, usually without causing a problem. It has been theorized that P. ovale metabolizes excess oil on scalp, which results in the formation of lower molecular weight fatty acids that cause skin irritation leading to dandruff. Although the present inventor has not studied the effect of the compounds of the present invention on dandruff itself, the compounds of the present invention have been found to reduce the excess sebum or oil on scalp.

Additional cosmetically or pharmaceutically beneficial ingredients can also be included in the formulated compositions of the present invention, which can be selected from skin cleansers, cationic, anionic surfactants, non-ionic surfactants, amphoteric surfactants, and zwitterionic surfactants, skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

In another preferred aspect, the cosmetically acceptable composition further comprises one or more excipient selected from the group consisting of water, saccharides, surface active agents, humectants, petrolatum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain amines from about C.sub.10 to C.sub.22, long chain fatty amines from about C.sub.10 to C.sub.22, fatty alcohols, ethoxylated fatty alcohols and di-tail phospholipids.

Representative saccharides include nonionic or cationic saccharides such as agarose, amylopectins, amyloses, arabinans, arabinogalactans, arabinoxylans, carageenans, gum arabic, carboxymethyl guar gum, carboxymethyl(hydroxypropyl) guar gum, hydroxyethyl guar gum, carboxymethyl cellulose, cationic guar gum, cellulose ethers including methyl cellulose, chondroitin, chitins, chitosan, chitosan pyrrolidone carboxylate, chitosan glycolate chitosan lactate, cocodimonium hydroxypropyl oxyethyl cellulose, colominic acid ([poly-N acetyl-neuraminic acid]), corn starch, curdlan, dermatin sulfate, dextrans, furcellarans, dextrans, cross-linked dextrans, dextrin, emulsan, ethyl hydroxyethyl cellulose, flaxseed saccharide (acidic), galactoglucomannans, galactomannans, glucomannans, glycogens, guar gum, hydroxy ethyl starch, hydroxypropyl methyl cellulose, hydroxy ethyl cellulose, hydroxy propyl cellulose, hydroxypropyl starch, hydroxypropylated guar gums, gellan gum, gellan, gum ghatti, gum karaya, gum tragancanth (tragacanthin), heparin, hyaluronic acid, inulin, keratin sulfate, konjac mannan, modified starches, laminarans, laurdimonium hydroxypropyl oxyethyl cellulose, okra gum, oxidized starch, pectic acids, pectin, polydextrose, polyquaternium-4, polyquaternium-10, polyquaternium-28, potato starch, protopectins, psyllium seed gum, pullulan, sodium hyaluronate, starch diethylaminoethyl ether, steardimonium hydroxyethyl cellulose, raffinose, rhamsan, tapioca starch, whelan, levan, scleroglucan, sodium alginate, stachylose, succinoglycan, wheat starch, xanthan gum, xylans, xyloglucans, and mixtures thereof. Microbial saccharides can be found in Kirk-Othmer Encyclopedia of Chemical Technology, Fourth Edition, Vol. 16, John Wiley and Sons, NY pp. 578-611 (1994), which is incorporated entirely by reference. Complex carbohydrates found in Kirk-Othmer Encyclopedia of Chemical Technology, Fourth Edition, Vol. 4, John Wiley and Sons, NY pp. 930-948, 1995 which is herein incorporated by reference.

The cosmetically acceptable composition of this invention may include surface-active agents. Surface-active agents include surfactants, which typically provide detersive functionality to a formulation or act simply as wetting agents. Surface-active agents can generally be categorized as anionic surface-active agents, cationic surface-active agents, nonionic surface-active agents, amphoteric surface-active agents and zwitterionic surface-active agents, and dispersion polymers.

Anionic surface-active agents useful herein include those disclosed in U.S. Pat. No. 5,573,709, incorporated herein by reference. Examples include alkyl and alkyl ether sulfates. Specific examples of alkyl ether sulfates which may be used In this invention are sodium and ammonium salts of lauryl sulfate, lauryl ether sulfate, coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 12 to about 16 carbon atoms and an average degree of ethoxylation of from about 1 to about 6 moles of ethylene oxide.

Another suitable class of anionic surface-active agents is the alkyl sulfuric acid salts. Important examples are the salts of an organic sulfuric acid reaction product of a hydrocarbon of the methane series, including iso-, neo-, and n-paraffins, having about 8 to about 24 carbon atoms, preferably about 12 to about 18 carbon atoms and a sulfonating agent, for example, sulfur trioxide or oleum, obtained according to known sulfonation methods, including bleaching and hydrolysis. Preferred are alkali metal and ammonium sulfated C.sub.12-38 n-paraffins.

Additional synthetic anionic surface-active agents include the olefin sulfonates, the beta-alkyloxy alkane sulfonates, and the reaction products of fatty acids esterified with isethionic acid and neutralized with sodium hydroxide, as well as succinamates. Specific examples of succinamates include disodium N-octadecyl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; dioctyl esters of sodium sulfosuccinic acid.

Preferred anionic surface-active agents for use in the cosmetically acceptable composition of this invention include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecyl benzene sulfonate.

Amphoteric surface-active agents which may be used in the cosmetically acceptable composition of this invention include derivatives of aliphatic secondary and tertiary amines, in which the aliphatic substituent contains from about 8 to 18 carbon atoms and an anionic water solubilizing group e.g., carboxy, sulfonate, sulfate, phosphate, or phosphonate. Representative examples include sodium 3-dodecyl-aminopropionate, sodium 3-dodecylaminopropane sulfonate, sodium lauryl sarcosinate, N-alkyltaurines such as the one prepared by reacting dodecylamine with sodium isethionate as described in U.S. Pat. No. 2,658,072, N-higher alkyl aspartic acids as described in U.S. Pat. No. 2,438,091, and the products sold under the trade name MIRANOL. as described in U.S. Pat. No. 2,528,378. Other sarcosinates and sarcosinate derivatives can be found in the CTFA Cosmetic Ingredient Handbook, Fifth Edition, 1988, page 42 incorporated herein by reference.

Quaternary ammonium compounds can also be used in the cosmetically acceptable composition of this invention as long as they are compatible in the compositions of the invention, wherein the structure is provided in the CTFA Cosmetic Ingredient Handbook, Fifth Edition, 1988, page 40. Cationic surface-active agents generally include, but are not limited to fatty quaternary ammonium compounds containing from about 8 to about 18 carbon atoms. The anion of the quaternary ammonium compound can be a common ion such as chloride, ethosulfate, methosulfate, acetate, bromide, lactate, nitrate, phosphate, or tosylate and mixtures thereof. The long chain alkyl groups can include additional or replaced carbon or hydrogen atoms or ether linkages. Other substitutions on the quaternary nitrogen can be hydrogen, hydrogen, benzyl or short chain alkyl or hydroxyalkyl groups such as methyl, ethyl, hydroxymethyl or hydroxyethyl, hydroxypropyl or combinations thereof.

Examples of quaternary ammonium compounds include but are not limited to: Behentrimonium chloride, Cocotrimonium chloride, Cethethyldimonium bromide, Dibehenyidimonium chloride, Dihydrogenated tallow benzylmonium chloride, disoyadimonium chloride, Ditallowdimonium chloride, Hydroxycetyl hydroxyethyl dimonium chloride, Hydroxyethyl Behenamidopropyl dimonium chloride, Hydroxyethyl Cetyidimonium chloride, Hydroxyethyl tallowdimonium chloride, myristalkonium chloride, PEG-2 Oleamonium chloride, PEG-5 Stearmonium chloride, PEG-15 cocoyl quaternium 4, PEG-2 stearalkonium 4, lauryltrimonium chloride; Quaternium-16; Quaternium-18, lauralkonium chloride, olealkmonium chloride, cetylpyridinium chloride, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dilauryidimonium chloride, cetalkonium chloride, dicetyidimonium chloride, soyatrimonium chloride, stearyl octyl dimonium methosulfate, and mixtures thereof. Other quaternary ammonium compounds are listed in the CTFA Cosmetic Ingredient Handbook, First Edition, on pages 41-42, incorporated herein by reference.

The cosmetically acceptable compositions that contain sebum reducing agents of the present invention may include long chain fatty amines from about C.sub.10 to C.sub.22 and their derivatives. Specific examples include dipalmitylamine, lauramidopropyidimethylamine, and stearamidopropyl dimethylamine. The cosmetically acceptable compositions of this invention may also include fatty alcohols (typically monohydric alcohols), ethoxylated fatty alcohols, and di-tail phospholipids, which can be used to stabilize emulsion or dispersion forms of the cosmetically acceptable compositions. They also provide a cosmetically acceptable viscosity. Selection of the fatty alcohol is not critical, although those alcohols characterized as having fatty chains of C.sub.10 to C.sub.32, preferably C.sub.14 to C.sub.22, which are substantially saturated alkanols will generally be employed. Examples include stearyl alcohol, cetyl alcohol, cetostearyl alcohol, myristyl alcohol, behenyl alcohol, arachidic alcohol, isostearyl alcohol, and isocetyl alcohol. Cetyl alcohol is preferred and may be used alone or in combination with other fatty alcohols, preferably with stearyl alcohol. When used the fatty alcohol is preferably included in the formulations of this invention at a concentration within the range from about 1 to about 8 weight percent, more preferably about 2 to about 6 weight percent. The fatty alcohols may also be ethoxylated. Specific examples include cetereth-20, steareth-20, steareth-21, and mixtures thereof. Phospholipids such as phosphatidylserine and phosphatidylcholine, and mixtures thereof may also be included. When used, the fatty alcohol component is included in the formulations at a concentration of about 1 to about 10 weight percent, more preferably about 2 to about 7 weight percent.

Nonionic surface-active agents, which can be used in the cosmetically acceptable composition of the present invention, include those broadly defined as compounds produced by the condensation of alkylene oxide groups (hydrophilic in nature) with an organic hydrophobic compound, which may be aliphatic or alkyl aromatic in nature. Examples of preferred classes of nonionic surface-active agents are: the long chain alkanolamides; the polyethylene oxide condensates of alkyl phenols; the condensation product of aliphatic alcohols having from about 8 to about 18 carbon atoms, in either straight chain or branched chain configuration, with ethylene oxide; the long chain tertiary amine oxides; the long chain tertiary phosphine oxides; the long chain dialkyl sulfoxides containing one short chain alkyl or hydroxy alkyl radical of from about 1 to about 3 carbon atoms; and the alkyl polysaccharide (APS) surfactants such as the alkyl polyglycosides; the polyethylene glycol (PEG) glyceryl fatty esters.

Zwitterionic surface-active agents such as betaines can also be useful in the cosmetically acceptable composition of this invention. Examples of betaines useful herein include the high alkyl betaines, such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH.sub.2).sub.3 radical is attached to the nitrogen atom of the betaine are also useful in this invention.

The anionic, cationic, nonionic, amphoteric or zwitterionic surface-active agents used in the cosmetically acceptable composition of this invention are typically used in an amount from about 0.1 to 50 percent by weight, preferably from about 0.5 to about 40 percent by weight, more preferably from about 1 to about 20 percent by weight.

The cosmetically acceptable compositions that contain sebum reducing agents of the present invention may include humectants, which act as hygroscopic agents, increasing the amount of water absorbed, held and retained. Suitable humectants for the formulations of this invention include but are not limited to: acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, sodium PCA, and combinations thereof. Glycerin is a particularly preferred humectant. The humectant is present in the composition at concentrations of from about 0.5 to about 40 percent by weight, preferably from about 0.5 to about 20 percent by weight and more preferably from about 0.5 to about 12 percent by weight.

The cosmetically acceptable compositions that contain sebum reducing agents of the present invention may include petrolatum or mineral oil components, which when selected will generally be USP or NF grade. The petrolatum may be white or yellow. The viscosity or consistency grade of petrolatum is not narrowly critical. Petrolatum can be partially replaced with mixtures of hydrocarbon materials, which can be formulated to resemble petrolatum in appearance and consistency. For example, mixtures of petrolatum or mineral oil with different waxes and the like may be combined. Preferred waxes include bayberry wax, candelilla wax, ceresin, jojoba butter, lanolin wax, montan wax, ozokerite, polyglyceryl-3-beeswax, polyglyceryl-6-pentastearate, microcrystalline wax, paraffin wax, isoparaffin, vaseline solid paraffin, squalene, oligomer olefins, beeswax, synthetic candelilla wax, synthetic carnauba, synthetic beeswax and the like may be blended together. Alkylmethyl siloxanes with varying degrees of substitution can be used to increase water retained by the skin. Siloxanes such as stearyl dimethicone, known as 2503 Wax, C30-45 alkyl methicone, known as AMS-C30 wax, and stearoxytrimethylsilane (and) stearyl alcohol, known as 580 Wax, each available from Dow Corning, Midland, Mich., USA. Additional alkyl and phenyl silicones may be employed to enhance moisturizing properties. Resins such as dimethicone (and) trimethylsiloxysilicate or Cyclomethicone (and) Trimethylsiloxysilicate fluid, may be utilized to enhance film formation of skin care products. When used, the petrolatum, wax or hydrocarbon or oil component is included in the formulations at a concentration of about 1 to about 20 weight percent, more preferably about 1 to about 12 weight percent. When used, the silicone resins can be included from about 0.1 to about 10.0 weight percent.

Emollients are defined as agents that help maintain the soft, smooth, and pliable appearance of skin. Emollients function by their ability to remain on the skin surface or in the stratum corneum. The cosmetically acceptable composition of this invention may include fatty ester emollients, which are listed in the International Cosmetic Ingredient Dictionary, Eighth Edition, 2000, p. 1768 to 1773. Specific examples of suitable fatty esters for use in the formulation of this invention include isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, PPG-5-Ceteth-20, 2-ethylhexyl isononoate, 2-ethylhexyl stearate, C.sub.12 to C.sub.16 fatty alcohol lactate, isopropyl lanolate, 2-ethyl-hexyl salicylate, and mixtures thereof. The presently preferred fatty esters are isopropyl myristate, isopropyl palmitate, PPG-5-Ceteth-20, and caprylic/capric triglycerides. When used the fatty ester emollient is preferably included in the formulations of this invention at a concentration of about 1 to about 8 weight percent, more preferably about 2 to about 5 weight percent.

The compositions that contain sebum reducing agents of the present invention may also include silicone compounds. Preferably, the viscosity of the silicone component is from about 0.5 to about 12,500 cps. Examples of suitable materials are dimethylpolysiloxane, diethylpolysiloxane, dimethylpolysiloxane-diphenylpolysiloxane, cyclomethicone, trimethylpolysiloxane, diphenylpolysiloxane, and mixtures thereof. Dimethicone, a dimethylpolysiloxane endblocked with trimethyl units, is one preferred example. Dimethicone having a viscosity between 50 and 1,000 cps is particularly preferred. When used, the silicone oils are preferably included in the formulations of this invention at a concentration of 0.1 to 5 weight percent, more preferably 1 to 2 weight percent.

The cosmetically acceptable compositions that contain sebum reducing agents of the present invention may include volatile and non-volatile silicone oils or fluids. The silicone compounds can be either linear or cyclic polydimethylsiloxanes with a viscosity from about 0.5 to about 100 centistokes. The most preferred linear polydimethylsiloxane compounds have a range from about 0.5 to about 50 centistokes. One example of a linear, low molecular weight, volatile polydimethylsiloxane is octamethyltrisiloxane. 200 fluid having a viscosity of about 1 centistoke. When used, the silicone oils are preferably included in the formulations of this invention at a concentration of 0.1 to 30 weight percent, more preferably 1 to 20 weight percent.

The cosmetically acceptable compositions that contain sebum reducing agents of the present invention may include volatile, cyclic, low molecular weight polydimethylsiloxanes (cyclomethicones). The preferred cyclic volatile siloxanes can be polydimethyl cyclosiloxanes having an average repeat unit of 4 to 6, and a viscosity from about 2.0 to about 7.0 centistokes, and mixtures thereof. Preferred cyclomethicones are available from Dow Corning, Midland, Mich., and from General Electric, Waterford, N.Y., USA. When used, the silicone oils are preferably included in the formulations of this invention at a concentration of 0.1 to 30 weight percent, more preferably 1 to 20 weight percent.

Silicone surfactants or emulsifiers with polyoxyethylene or polyoxypropylene side chains may also be used in compositions of the current invention. Preferred examples include dimethicone copolyols and 5225C Formulation Aids, available from Dow Corning, Midland, Mich., USA and Silicone SF-1528, available from General Electric, Waterford, N.Y., USA. The side chains may also include alkyl groups such as lauryl or cetyl. Preferred are lauryl methicone copolyol. 5200 Formulation Aid, and cetyl dimethicone copolyol, known as Abil EM-90, available from Goldschmidt Chemical Corporation, Hopewell, Va. Also preferred is lauryl dimethicone, known as Belsil LDM 3107 VP, available from Wacker-Chemie, Munchen, Germany. When used, the silicone surfactants are preferably included in the formulations of this invention at a concentration of 0.1 to 30 weight percent, more preferably 1 to 15 weight percent. Amine functional silicones and emulsions may be utilized in the present invention. Preferred examples include Dow Corning 8220, Dow Corning 939, Dow Corning 949, Dow Corning 2-8194, all available from Dow Corning, Midland, Mich., USA. Also preferred is Silicone SM 253 available from General Electric, Waterford, N.Y., USA. When used, the amine functional silicones are preferably included in the formulations of this invention at a concentration of 0.1 to 5 weight percent, more preferably 0.1 to 2.0 weight percent.

The cosmetically acceptable compositions that contain sebum reducing agents of the present invention may include volatile hydrocarbon oils. The volatile hydrocarbon comprises from about C.sub.6 to C.sub.22 atoms. A preferred volatile hydrocarbon is an aliphatic hydrocarbon having a chain length from about C.sub.6 to C.sub.16 carbon atoms. An example of such compound includes isohexadecane, under the tradename Permethyl 101A, available from Presperse, South Plainfield, N.J., USA. Another example of a preferred volatile hydrocarbon is C.sub.12 to C.sub.14 isoparaffin, under the tradename Isopar M, available from Exxon, Baytown, Tex., USA. When used, the volatile hydrocarbons are preferably included in the formulations of this invention at a concentration of 0.1 to 30 weight percent, more preferably 1 to 20 weight percent.

The cosmetically acceptable compositions that contain sebum reducing agents of the present invention may include cationic and ampholytic conditioning polymers. Examples of such include, but are not limited to those listed by the International Cosmetic Ingredient Dictionary published by the Cosmetic, Toiletry, and Fragrance Association (CTFA), 1110 17 Street, N.W., Suite 300, Washington, D.C. 20036. General examples include quaternary derivatives of cellulose ethers, quaternary derivatives of guar, homopolymers and copolymers of DADMAC, homopolymers and copolymers of MAPTAC and quaternary derivatives of starches. Specific examples, using the CTFA designation, include, but are not limited to Polyquaternium-10, Guar hydroxypropyltrimonium chloride, Starch hydroxypropyltrimonium chloride, Polyquaternium-4, Polyquaternium-5, Polyquaternium-6, Polyquaternium-7, Polyquaternium-14, Polyquaternium-15, Polyquaternium-22, Polyquaternium-24, Polyquaternium-28, Polyquaternium-32, Polyquaternium-33, Polyquaternium-36, Polyquaternium-37, Polyquaternium-39, Polyquaternium-45, Polyquaternium-47 and polymethacrylamidopropyltrimonium chloride, and mixtures thereof. When used, the conditioning polymers are preferably included in the cosmetically acceptable composition of this invention at a concentration of from 0.1 to 10 weight percent, preferably from 0.2 to 6 weight percent and most preferably from 0.2 to 5 weight percent.

The cosmetically acceptable compositions that contain sebum reducing agents of the present invention may include one or more rheological modifiers. The rheological modifiers that can be used in this invention include, but are not limited to high molecular weight crosslinked homopolymers of acrylic acid, and Acrylates/C10-30 Alkyl Acrylate Crosspolymer, such as the Carbopol. and Pemulen series, both available from B. F. Goodrich, Akron, Ohio, USA; anionic acrylate polymers such as Salcare and cationic acrylate polymers such as Salcare SC96, available from Ciba Specialties, High Point, N.C., USA; Acrylamidopropylttrimonium chloride/acrylamide; Hydroxyethyl methacrylates polymers, Steareth-10 Allyl Ether/Acrylate Copolymer; Acrylates/Beheneth-25 Metacrylate Copolymer, known as Aculyn, available from International Specialties, Wayne, N.J., USA; Glyceryl Polymethacrylate, Acrylates/Steareth-20 Methacrylate Copolymer; bentonite; gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; polyvinyl alcohol, PVM/MA copolymer, PVM/MA decadiene crosspolymer, poly(ethylene oxide) based thickeners, sodium carbomer, and mixtures thereof. When used, the rheology modifiers are preferably included in the cosmetically acceptable composition of this invention at a concentration of from 0.01 to 12 weight percent, preferably from 0.05 to 10 weight percent and most preferably from 0.1 to 6 weight percent.

The cosmetically acceptable composition that contain sebum reducing agents of the present invention may include one or more antioxidants, which include, but are not limited to ascorbic acid, BHT, BHA, erythorbic acid, bisulfite, thioglycolate, tocopherol, sodium metabisulfite, vitamin E acetate, and ascorbyl palmitate. The anti oxidants will be present at from 0.01 to 5 weight percent, preferably 0.1 to 3 weight percent and most preferably from 0.2 to 2 weight percent of the cosmetically acceptable composition.

The cosmetically acceptable compositions that contain sebum reducing agents of the present invention may include one or more sunscreen active agents. Examples of sunscreen active agents include, but are not limited to octyl methoxycinnamate (ethylhexyl p-methoxycinnamate), octyl salicylate oxybenzone (benzophenone-3), benzophenone-4, menthyl anthranilate, dioxybenzone, aminobenzoic acid, amyl dimethyl PABA, diethanolamine p-methoxy cinnamate, ethyl 4-bis (hydroxypropyl) aminobenzoate, 2-ethylhexy 1-2-cyano-3,3-diphenylacrylate, homomenthyl salicylate, glyceryl aminobenzoate, dihydroxyacetone, octyl dimethyl PABA, 2-phenylbenzimidazole-5-sulfonic acid, triethanolamine salicylate, zinc oxide, and titanium oxide, and mixtures thereof. The amount of sunscreen used in the cosmetically acceptable composition of this invention will vary depending on the specific UV absorption wavelength(s) of the specific sunscreen active(s) used and can be from 0.1 to 10 percent by weight, from 2 to 8 percent by weight.

The cosmetically acceptable compositions that contain sebum reducing agents of the present invention may include one or more preservatives. Example of preservatives, which may be used include, but are not limited to 1,2-dibromo-2,4-dicyano butane (Methyldibromo Glutaronitrile, known as MERGUARD. Nalco Chemical Company, Naperville, Ill., USA), benzyl alcohol, imidazolidinyl urea, 1,3-bis (hydroxymethyl)-5,5-dimethyl-2,3-imidazolidinedione (e.g., DMDM Hydantoin, known as GLYDANT, Lonza, Fairlawn, N.J., USA.), methylchloroisothiazolinone and methylisothiazolinone (e.g., Kathon, Rohm & Haas Co., Philadelphia, Pa., USA), methyl paraben, propyl paraben, phenoxyethanol, and sodium benzoate, and mixtures thereof.

The cosmetically acceptable compositions that contain sebum reducing agents of the present invention may include any other ingredient by normally used in cosmetics. Examples of such ingredients include, but are not limited to buffering agents, fragrance ingredients, chelating agents, color additives or dyestuffs which can serve to color the composition itself or keratin, sequestering agents, softeners, foam synergistic agents, foam stabilizers, sun filters and peptizing agents.

The surface of pigments, such titanium dioxide, zinc oxide, talc, calcium carbonate or kaolin, can be treated with the unsaturated quaternary ammonium compounds described herein and then used in the cosmetically acceptable composition of this invention. The treated pigments are then more effective as sunscreen actives and for use in color cosmetics such as make up and mascara.

The cosmetically acceptable compositions that contain sebum reducing agents of the present invention can be presented in various forms. Examples of such forms include, but are not limited a solution, liquid, cream, emulsion, dispersion, gel, thickening lotion.

The cosmetically acceptable compositions that contain sebum reducing agents of the present invention may contain water and also any cosmetically acceptable solvent. Examples of acceptable solvents include, but are not limited to monoalcohols, such as alkanols having 1 to 8 carbon atoms (like ethanol, isopropanol, benzyl alcohol and phenylethyl alcohol) polyalcohols, such as alkylene glycols (like glycerine, ethylene glycol and propylene glycol) and glycol ethers, such as mono-, di- and tri-ethylene glycol monoalkyl ethers, for example ethylene glycol monomethyl ether and diethylene glycol monomethyl ether, used singly or in a mixture. These solvents can be present in proportions of up to as much as 70 percent by weight, for example from 0.1 to 70 percent by weight, relative to the weight of the total composition.

The cosmetically acceptable compositions that contain sebum reducing agents of the present invention can also be packaged as an aerosol, in which case it can be applied either in the form of an aerosol spray or in the form of an aerosol foam. As the propellant gas for these aerosols, it is possible to use, in particular, dimethyl ether, carbon dioxide, nitrogen, nitrous oxide, air and volatile hydrocarbons, such as butane, isobutane, and propane.

The cosmetically acceptable compositions that contain sebum reducing agents of the present invention can also can contain electrolytes, such as aluminum chlorohydrate, alkali metal salts, e.g., sodium, potassium or lithium salts, these salts preferably being halides, such as the chloride or bromide, and the sulfate, or salts with organic acids, such as the acetates or lactates, and also alkaline earth metal salts, preferably the carbonates, silicates, nitrates, acetates, gluconates, pantothenates and lactates of calcium, magnesium and strontium.

Compositions for treating skin that contain sebum reducing agents of the present invention include leave-on or rinse-off skin care products such as lotions, hand/body creams, shaving gels or shaving creams, body washes, sunscreens, liquid soaps, deodorants, antiperspirants, suntan lotions, after sun gels, bubble baths, hand or mechanical dishwashing compositions, and the like. In addition to the polymer, skin care compositions may include components conventionally used in skin care formulations. Such components include for example; (a) humectants, (b) petrolatum or mineral oil, (c) fatty alcohols, (d) fatty ester emollients, (e) silicone oils or fluids, and (f) preservatives. These components must in general be safe for application to the human skin and must be compatible with the other components of the formulation. Selection of these components is generally within the skill of the art. The skin care compositions may also contain other conventional additives employed in cosmetic skin care formulations. Such additives include aesthetic enhancers, fragrance oils, dyes and medicaments such as menthol and the like.

The skin care compositions that contain sebum reducing agents of the present invention may be prepared as oil-in-water, water-in-oil emulsions, triple emulsions, or dispersions.

Preferred oil-in-water emulsions are prepared by first forming an aqueous mixture of the water-soluble components, e.g. unsaturated quaternary ammonium compounds, humectants, water-soluble preservatives, followed by adding water-insoluble components. The water-insoluble components include the emulsifier, water-insoluble preservatives, petrolatum or mineral oil component, fatty alcohol component, fatty ester emollient, and silicone oil component. The input of mixing energy will be high and will be maintained for a time sufficient to form a water-in-oil emulsion having a smooth appearance (indicating the presence of relatively small micelles in the emulsion). Preferred dispersions are generally prepared by forming an aqueous mixture of the water-soluble components, followed by addition of thickener with suspension power for water-insoluble materials.

Compositions that contain sebum reducing agents of the present invention for treating hair include bath preparations such as bubble baths, soaps, and oils, shampoos, conditioners, hair bleaches, hair coloring preparations, temporary and permanent hair colors, color conditioners, hair lighteners, coloring and non-coloring hair rinses, hair tints, hair wave sets, permanent waves, curling, hair straighteners, hair grooming aids, hair tonics, hair dressings and oxidative products. The dispersion polymers may also be utilized in styling type leave-in products such as gels, mousses, spritzes, styling creams, styling waxes, pomades, balms, and the like, either alone or in combination with other polymers or structuring agents in order to provide control and hair manageability with a clean, natural, non-sticky feel.

Hair care compositions of this invention give slippery feel and that can be easily rinsed from the hair due to the presence of the dispersion polymer, volatile silicones, other polymers, surfactants or other compounds that may alter the deposition of materials upon the hair.

In the case of cleansing formulations such as a shampoo for washing the hair, or a liquid hand soap, or shower gel for washing the skin, the compositions that contain sebum reducing agents of the present invention contain anionic, cationic, nonionic, zwitterionic or amphoteric surface-active agents typically in an amount from about 3 to about 50 percent by weight, preferably from about 3 to about 20 percent, and their pH is general in the range from about 3 to about 10.

Preferred shampoos of this invention contain combinations of anionic surfactants with zwitterionic surfactants and/or amphoteric surfactants. Especially preferred shampoos contain from about 0 to about 16 percent active of alkyl sulfates, from 0 to about 50 weight percent of ethoxylated alkyl sulfates, and from 0 to about 50 weight percent of optional surface-active agents selected from the nonionic, amphoteric, and zwitterionic surface-active agents, with at least 5 weight percent of either alkyl sulfate, ethoxylated alkyl sulfate, or a mixture thereof, and a total surfactant level of from about 10 weight to about 25 percent.

The shampoo for washing hair also can contain other conditioning additives such as silicones and conditioning polymers typically used in shampoos. U.S. Pat. No. 5,573,709 provides a list of non-volatile silicone conditioning agents that can be used in shampoos. The conditioning polymers for use with the present invention are listed in the Cosmetic, Toiletries and Fragrance Associations (CTFA) dictionary. Specific examples include the Polyquaterniums (example Polyquaternium-1 to Polyquaternium-50), Guar Hydroxypropyl Trimonium Chloride, Starch Hydroxypropyl Trimonium Chloride and Polymethacrylamidopropyl Trimonium Chloride.

Other preferred embodiments consist of use in the form of a rinsing lotion to be applied mainly before or after shampooing. These lotions typically are aqueous or aqueous-alcoholic solutions, emulsions, thickened lotions or gels. If the compositions are presented in the form of an emulsion, they can be nonionic, anionic or cationic. The nonionic emulsions consist mainly of a mixture of oil and/or a fatty alcohol with a polyoxyethyleneated alcohol, such as polyoxyethyleneated stearyl or cetyl/stearyl alcohol, and cationic surface-active agents can be added to these compositions. The anionic emulsions are formed essentially from soap.

If the compositions that contain sebum reducing agents of the present invention are presented in the form of a thickened lotion or a gel, they contain thickeners in the presence or absence of a solvent. The thickeners which can be used are especially resins, Carbopol-type acrylic acid thickeners available from B.F. Goodrich; xanthan gums; sodium alginates; gum arabic; cellulose derivatives and poly-(ethylene oxide) based thickeners, and it is also possible to achieve thickening by means of a mixture of polyethylene glycol stearate or distearate or by means of a mixture of a phosphoric acid ester and an amide. The concentration of thickener is generally 0.05 to 15 percent by weight. If the compositions are presented in the form of a styling lotion, shaping lotion, or setting lotion, they generally comprise, in aqueous, alcoholic or aqueous-alcoholic solution, the ampholyte polymers defined above.

In the case of hair fixatives, the composition that contain sebum reducing agents of the present invention may also contain one or more additional hair fixative polymers. When present, the additional hair fixative polymers are present in a total amount of from about 0.25 to about 10 percent by weight. The additional hair fixative resin can be selected from the following group as long as it is compatible with a given dispersion polymer: acrylamide copolymer, acrylamide/sodium acrylate copolymer, acrylate/ammonium methacrylate copolymer, an acrylate copolymer, an acrylic/acrylate copolymer, adipic acid/dimethylaminohydroxypropyl diethylenetriamine copolymer, adipic acid/epoxypropyl diethylenetriamine copolymer, allyl stearate/VA copolymer, aminoethylacrylate phosphate/acrylate copolymer, an ammonium acrylate copolymer, an ammonium vinyl acetate/acrylate copolymer, an AMP acrylate/diacetoneacrylamide copolymer, an AMPD acrylate/diacetoneacrylamide copolymer, butyl ester of ethylene/maleic anhydride copolymer, butyl ester of PVM/MA copolymer, calcium/sodium PVM/MA copolymer, corn starch/acrylamide/sodium acrylate copolymer, diethylene glycolamine/epichlorohydrin/piperazine-copolymer, dodecanedioic acid/cetearyl alcohol/glycol copolymer, ethyl ester of PVM/MA copolymer, isopropyl ester of PVM/MA copolymer, karaya gum, a methacryloyl ethyl betaine/methacrylate copolymer, an octylacrylamide/acrylate/butylaminoethyl methacrylate copolymer, an octylacrylamide/acrylate copolymer, phthalic anhydride/glycerin/glycidyl decanoate copolymer, a phthalic/trimellitic/glycol copolymer, polyacrylamide, polyacrylamidomethylpropane sulfonic acid, polybutylene terephthalate, polyethylacrylate, polyethylene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-39, polyquaternium-47, polyvinyl acetate, polyvinyl butyral, polyvinyl imidazolinium acetate, polyvinyl methyl ether, PVM/MA copolymer, PVP, PVP/dimethylaminoethylmethacrylate copolymer, PVP/eicosene copolymer, PVP/ethyl methacrylate/methacrylic acid copolymer, PVP/hexadecene copolymer, PVP/VA copolymer, PVP/vinyl acetate/itaconic acid copolymer, shellac, sodium acrylates copolymer, sodium acrylates/Acrylnitrogens copolymer, sodium acrylate/vinyl alcohol copolymer, sodium carrageenan, starch diethylaminoethyl ether, stearylvinyl ether/maleic anhydride copolymer, sucrose benzoate/sucrose acetate isobutyrate/butyl benzyl phthalate copolymer, sucrose benzoate/sucrose acetate isobutyrate/butyl benzyl phthalate/methyl methacrylate copolymer, sucrose benzoate/sucrose acetate isobutyrate copolymer, a vinyl acetate/crotonate copolymer, vinyl acetate/crotonic acid copolymer, vinyl acetate/crotonic acid/methacryloxybenzophenone-1 copolymer, vinyl acetate/crotonic acid/vinyl neodecanoate copolymer, and mixtures thereof. Synthetic polymers used for creating styling aids are described in “The History of Polymers in Haircare,” Cosmetics and Toiletries, 103 (1988), incorporated herein by reference. Other synthetic polymers that may be used with the present invention can be referenced in the CTFA Dictionary, Fifth Edition, 2000, incorporated herein by reference.

The cosmetically acceptable carrier contained in the cosmetic compositions that contain sebum reducing agents of the present invention may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonites, silica, talc, zinc oxide or mixtures of these ingredients.

In the formulation of powder or spray, it may comprise lactose, talc, silica, aluminum hydroxide, calcium silicate, polyamide powder and mixtures of these ingredients. Spray may additionally comprise the customary propellants, for example, chlorofluorohydrocarbons, propane, butane, diethyl ether, or dimethyl ether.

The formulation of solution and emulsion that contain sebum reducing agents of the present invention may comprise solvent, solubilizer and emulsifier, for example water, ethanol, isopropanol, ethyl carbonate, ethyl acetate, benzyl alcohol, benzyl benzoate, propylene glycol, 1,3-butyleneglycol, oils, in particular cottonseed oil, groundnut oil, maize germ oil, olive oil, castor oil and sesame seed oil, glycerol fatty esters, polyethylene glycol and fatty acid esters of sorbitan or mixtures of these ingredients.

The formulation of suspension that contain sebum reducing agents of the present invention may comprise liquid diluents, for example water, ethanol or propylene glycol, suspending agents, for example ethoxylated isosteary alcohols, polyoxyethylene sorbitol esters and poly oxyethylene sorbitan esters, micocrystalline cellulose, aluminum metahydroxide, bentonite, agar and tragacanth or mixtures of these ingredients.

The formulation of cleansing compositions that contain sebum reducing agents of the present invention with surfactant may comprise aliphatic alcohol sulfate, aliphatic alcohol ether sulfate, sulfosucinnate monoester, isothinate, imidazolium derivatives, methyltaurate, sarcocinate, fatty acid amide ether sulfate, alkyl amido betain, aliphatic alcohol, fatty acid glyceride, fatty acid diethanolamide, vegetable oil, lanoline derivatives, ethoxylated glycerol fatty acid ester or mixtures of these ingredients.

Additional antioxidant ingredients and compositions can be selected from, but not limited to, Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), Pyridoxine, Magnolol, Honokiol, Paeonol, Resacetophenone, Quinacetophenone, arbutin, kojic acid, and combinations thereof.

The blood micro-circulation improvement ingredients and compositions can be added to compositions that contain sebum reducing agents of the present invention. These are selected from Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis Vinifera) leaves, apigenin, phytosan, luteolin, and combinations thereof.

The anti-inflammatory ingredients can be added to compositions that contain sebum reducing agents of the present invention. These can be selected from at least one antioxidant class of Cyclo-oxygenase (for example, COX-1 or COX-2) or Lipoxygenase (for example, LOX-5) enzyme inhibitors such as Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, and Emblica extract. Anti-inflammatory composition can additionally be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof.

Certain divalent metal ions can be added to compositions that contain sebum reducing agents of the present invention. The examples of such metal ions include zinc, copper, manganese, vanadium, chromium, cobalt, and iron.

Determination of the Skin Surface Sebum.

The Measurement Principle. The measurement is based on grease-spot photometry. A special tape becomes transparent in contact with the sebum on the skin surface. For the determination of the sebum, the measuring head of the cassette is inserted into the aperture of the device (SEBUMETER SM 815), where the transparency is measured by a light source sending light through the tape, which is reflected by a little mirror behind the tape. A photocell measures the transparency. The light transmission represents the sebum content on the surface of the measuring area. A microprocessor calculates the result, which is shown on the display in microgram sebum/square cm of the skin.

For testing, 15 panelists were selected. Sample from Example 15 was applied on one cheek, one side of forehead, and one underarm of the panelists. Plain water was applied on the other cheek, the other side of, forehead, and the other underarm of he panelists. After 8 hours the amount of sebum was evaluated by Sebumeter. The % reduction (or increase) of sebum was calculated from the following equation

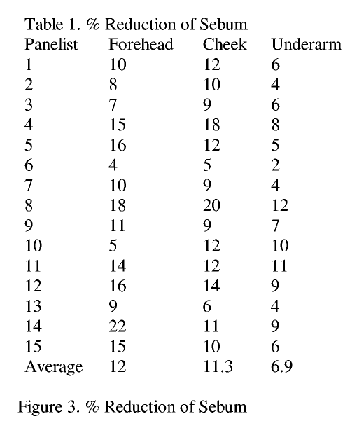

% Sebum Reduction (or Increase)=(A)+(B)/(B)×%; wherein A=amount of sebum where sample of Example 15 was applied, B=amount of sebum where plain water was applied. These data are presented in FIG. 3 (Table 1). These data clearly show average sebum reduction of 12.0%, 11.3%, and 6.9% in forehead, cheek, and underarm, respectively. In no panelist an increase of sebum was noted. FIG. 3.

### EXAMPLES

The following examples are presented to illustrate presently preferred practice thereof. These examples also include the formulation of consumer desirable lotion, cream, and other such compositions for their retail marketing. As illustrations they are not intended to limit the scope of the invention. All quantities are in weight %.

### Example 1

### Scalp Sebum Reduction Serum

Ingredients % Weight (1) Deionized water 20.0 (2) Tigogenin 5.0 (3) Methylpropanediol 69.5 (4) Dimethicone copolyol 4.0 (5) Preservatives 0.5 (6) Ammonium Acryloyldimethyltaurate/vp copolymer 1.0. Procedure. Make main batch by mixing (2) to (5) at room temperature. Pre-mix (1) and (6) to a clear paste and add to main batch with mixing. The product has a clear to slightly hazy syrup-like appearance, typical of a skin serum product. It is absorbed rapidly with a silky smooth skin feel.

### Example 2

### Skin Whitening Serum with Sebum Reduction

Ingredients % Weight (1) Deionized water 20.0 (2) Quinacetophenone 5.0 (3) Methylpropanediol 69.0 (4) Dimethicone copolyol 4.0 (5) Preservatives 0.5 (6) Protodioscin 0.5. (7) Ammonium Acryloyldimethyltaurate/VP copolymer 1.0 Procedure. Make main batch by mixing (2) to (6) at room temperature. Pre-mix (1) and (7) to a clear paste and add to main batch with mixing. The product has a clear to slightly hazy syrup-like light blue appearance, typical of a skin serum product. It is absorbed rapidly with a silky smooth skin feel.

### Example 3

### Antiaging, Sebum Reducing Cream.

Ingredients % Weight (1) Deionized water 79.5 (2) Cetearyl alcohol (and) dicetyl phosphate (and) Ceteth-10 phosphate 5.0 (3) Cetyl alcohol 2.0 (4) Glyceryl stearate (and) PEG-100 stearate 4.0 (5) Caprylic/capric triglyceride 5.0 (6) Resacetophenone 3.0 (7) Hecogenin 1.0 (8) (8) Preservatives 0.5. Procedure. Mix 1 to 5 and heat to 75-80° C. Adjust pH to 4.0 4.5. Cool to 35-40 C with mixing. Add 6 to 8 with mixing. Adjust pH to 4.0-4.5, if necessary. White to off-white cream.

### Example 4

### Collagen Boosting Sebum Control Facial Mask

Ingredient. % (1) Chitosan 5.0 (2) 2,5-Dihydroxy acetophenone 5.0 (3) Glycerin 17.7 (4) Water 70.6 (5) Protodioscin 0.5 (6) Zinc Salicylate 0.5 (7) Tigogenin 0.2 (8) Preservatives 0.5 Procedure: Mix 1, 2, and 3 to a paste. Mix 4 to 8 separately to a clear solution. Add this to main batch and mix. A clear gel product is obtained. It is applied on the face and neck and left for 10 to 30 minutes, then rinsed off.

### Example 5

### Age Spot and Sebum Reduction Cream

Ingredient % (1) Water 65.3 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Dioscin 0.5 (12) Shea butter 2.0 (13) Tigogenin 1.0 (14) Water 5.0 (15) Zinc Lactate 1.0 (16) Zinc Hydroxycitrate 3.1 (17) 2,4-Dihydroxy Acetophenone (Resacetophenone) 1.1 (18) Paeonol 1.5 (19) Carnosine 0.1 (20) Cyclomethicone, Dimethicone Crosspolymer 2.0 (21) Arbutin 0.5 (22) Polysorbate-20 2.0 (23) Sepigel-305 2.0. Procedure. Mix (1) to (13) and heat at 70 to 80 C till homogenous. Cool to 40 to 50 C. Premix (14) to (16) and add to batch with mixing. Add all other ingredients and mix. Cool to room temperature. An off-white cream is obtained.

### Example 6

### Acne Cream with Sebum Reduction

Ingredient % (1) Water 62.3 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Water 5.0 (15) Dioscin 4.0 (16) Niacinamide Hydroxycitrate 2.2 (17) 2,4-Dihydroxy Acetophenone (Resacetophenone) 1.1 (18) Hecogenin 1.5 (19) Carnosine 0.1 (20) Cyclomethicone, Dimethicone Crosspolymer 2.0 (21) Arbutin 0.5 (22) Pyridoxine Salicylate (23) Polysorbate-20 2.0 (24) Sepigel-305 2.0. Procedure. Mix (1) to (13) and heat at 70 to 80 C till homogenous. Cool to 40 to 50 C. Premix (14) to (16) and add to batch with mixing. Add all other ingredients and mix. Cool to room temperature. An off-white cream is obtained.

### Example 7

### Skin Brightening Cleanser with Sebum Reduction

Ingredient % (1) PEG-6 63.120 (2) Hydroxypropyl Cellulose 0.3 (3) Boswellia Serrata 0.05 (4) Sodium Cocoyl Isethionate 20.0 (5) Sodium Lauryl Sulfoacetate 5.0 (6) L-Glutathione 0.01 (7) Zinc Salicylate 0.11 (8) Protodioscin 0.1 (9) Tigogenin 0.11 (10) Ascorbic acid 10.0 (11) Phenoxyethanol 0.7 (12) Ethylhexylglycerin 0.3 (13) Fragrance 0.2. Procedure. Mix (1) and (2) to a clear thin gel. Add all other ingredients and mix in a homogenizer. A white cream-like cleanser is obtained.

### Example 8

### Antiaging Gel with Sebum Reduction

Ingredients % (1) Triethyl Citrate 67.75 (2) Ethylenediamine/Hydrogenated Dimer Dilinoleate Copolymer Bis-Di-C14-18 Alkyl Amide 10.0 (3) Ximenia Oil 0.1 (4) Tigogenin 0.25 (5) Magnolol (and Honokiol 0.2 (6) Protodioscin 0.5 (7) Tetrahydrocurcuminoids 0.2 (8) Zeolite 20.0 (9) Fragrance 1.0. Procedure. Mix (1) and (2) and heat at 80 to 90 C till clear. Cool to 40 to 50 C and add all other ingredients and mix. Cool to room temperature. A white gel-like product is obtained.

### Example 9

### Anti-Wrinkle Sebum Reduction Transparent Gel

Ingredients % (1) C12-15 Alkyl Benzoate 96.75 (2) Dibutyl Lauroyl Glutamide 1.0 (3) Ximenia Oil 0.1 (4) Tigogenin 0.25 (5) Magnolol (and Honokiol 0.2 (6) Paeonol 0.5 (7) Tetrahydrocurcuminoids 0.2 (8) Hecogenin 1.0. Procedure. Mix (1) and (2) and heat at 95 to 110 C till clear. Cool to 40 to 50 C and add all other ingredients and mix. Cool to room temperature. A transparent gel-like product is obtained.

### Example 10

### Sebum Reduction Spray Lotion with Anti-inflammatory Agents

Ingredients % (1) PEG-4 81.0 (2) Triethyl Citrate 16.0 (3) Fragrance 0.5 (4) Paeonol 0.5 (5) Acetyl Tigogenin 2.0. Procedure. Mix all ingredients till a clear solution is obtained. Fill in spray bottles.

### Example 11

### Anti-inflammatory Color-Changing Acne Mask with Sebum Reduction Agents in a Controlled Release Delivery System

Ingredients % (1) Grapeseed oil 34.28 (2) Ethylenediamine/Hydrogenated Dimer Dilinoleate Copolymer Bis-Di-C14-18 Alkyl Amide 5.0 (3) Hecogenin 2.0 (4) Propyl Paraben 0.3 (5) Jojoba oil 0.5 (6) Sweet Almond oil 4.0 (7) Shea butter 0.2 (8) Mango butter 0.2 (9) Avocado utter 0.2 (10) Murumuru butter 0.2 (11) Color Change Green/Blue dye 0.01 (12) Zinc Hydroxybenzoate 5.5 (13) Vitamin E 0.11 (14) Phenoxyethanol 0.7 (15) Zeolite 31.0 (16) Ethylhexylglycerin 0.5 (17) Laureth-3 15.0 (18) Fragrance 0.5. Procedure. Mix (1) to (10) and heat at 70 to 80 C till clear. Cool to 35 to 45 C and all other ingredients and mix. Cool to room temperature. A light green thin paste is obtained. Upon contact with water, it turns blue and releases heat.

### Example 12

### Sebum Reduction Shampoo

Ingredient % (1) Water 64.2 (2) Protodioscin 1.2 (3) Sodium Lauryl Sulfoacetate 10.0 (4) Disodium Laureth Sulfosuccinate 20.0 (5) Phenoxyethanol 0.7 (6) Chlorphenesin 0.3 (7) PEG-120 Methyl Glucose Dioleate 2.5. (8) Hydrolyzed Soy Protein 0.5 (9) Hydrolyzed Silk Protein 0.5 (10) Oat Extract 0.1. Procedure. Mix (1) to (7) and heat at 60 to 70 C to a clear solution. Cool to 35 to 40 C and add all other ingredients and mix. Cool to room temperature.

### Example 13

### Scalp Sebum Reduction Lotion

Ingredients % (1) Water 39.158 (2) Acrylates/C10-30 Alkyl Acrylate Crosspolymer 0.5 (3) Escin 0.1 (4) Sodium Stearyl Phthalamate 1.0 (5) Sodium Hydroxide 0.142 (6) Cetyl Alcohol 4.0 (7) Phenoxyethanol 0.7 (8) Chlorphenesin 0.3 (9) Triethyl Citrate 10.0 (10) Ethylhexylglycein 0.5 (11) Protodioscin 10.0 (12) PEG-6 2.0 (13) Tetrahydrocurcuminoids 0.1 (14) Magnolol 0.1 (15) Paeonol 0.2 (16) Fragrance 1.0. Procedure. Mix (1) to (11) and heat at 80 to 90 C till clear. Cool to 45 to 55. Pre-mix (12) to (16) and add to main batch and mix. Cool to room temperature and adjust pH to 7.5.

### Example 14

### Sebum Reduction Make-up Remover Fluid

Ingredients % (1) Water 39.158 (2) Acrylates/C10-30 Alkyl Acrylate Crosspolymer 0.5 (3) Hecogenin 0.1 (4) Sodium Stearyl Phthalamate 1.0 (5) Sodium Hydroxide 0.142 (6) Cetyl Alcohol 4.0 (7) Phenoxyethanol 0.7 (8) 1,2-Octanediol 0.3 (9) Triethyl Citrate 10.0 (10) Methyl Soyate 30.0 (11) Ethylhexylglycein 0.5 (12) Polysorbate-20 10.0 (13) PEG-6 2.0 (14) Tetrahydrocurcuminoids 0.1 (15) Magnolol 0.1 (16) Dioscin 0.2 (17) Fragrance 1.0. Procedure. Mix (1) to (12) and heat at 80 to 90 C till clear. Cool to 45 to 55. Pre-mix (13) to (16) and add to main batch and mix. Add (17) and mix. Cool to room temperature and adjust pH to 7.5.

### Example 15

### Test Solution of Protodioscin

Ingredients % (1) Water 98.0 (2) Protodiosin 2.0. Mix two ingredients at 40 to 50 C to a clear solution.

### BRIEF DESCRIPTION OF THE DRAWINGS

[FIG. 1] Structure of a Furostanol Saponin.

[FIG. 2] Structure of Furostanol Sapogenins.

[FIG. 3] % Reduction of Sebum.

What is claimed is:

1. A method for reducing sebum or oil on skin, comprising the application of a furostanol saponin or sapogenin.

2. The method of claim 1, wherein said saponin or sapogenin is present in a composition, said composition further comprising a physiologically acceptable medium.

3. The method of claim 1, wherein said saponin or sapogenin is selected from Dioscin, Diosgenin, Hecogenin, Heconin, Tigogenin, Tigonin, Gitogenin, Chlorogenin, Eruboside, Protoeruboside, Manogenin, Shlorogenin, Hainangenin, Protodioscin, Protodiosgenin, Aculeoside, Smilagenin, Sarsapogenin, Yamogenin, Yuccagenin, Gracillin, Sativoside, and combinations thereof.

4. The method of claim 1, wherein said saponin is Protodioscin.

5. The method of claim 1, wherein said sapogenin is Tigogenin.

6. The method of claim 1, wherein said sapogenin is Hecogenin.

7. The method of claim 1, wherein said saponin is Dioscin.

8. The method of claim 1, wherein said saponin is Diosgenin.

9. The method of claim 1, wherein said sebum or oily skin is associated with acne.

10. The method of claim 1, wherein said sebum or oily skin is associated with dandruff.

11. The method of claim 1, wherein said sebum or oily skin is associated with body malodor.

12. The method of claim 1, wherein said sebum or oily skin is associated with underarm malodor.

13. The method according to claim 1, wherein such saponin or sapogenin is present in a composition, and represents from 0.01 to 5.0% of the total weight of the composition, the composition further comprising a physiologically acceptable medium.

14. The method according to claim 3, comprising applying a plant extract that contains a saponin or sapogenin selected from the group consisting of Dioscin, Diosgenin, Hecogenin, Heconin, Tigogenin, Tigonin, Gitogenin, Chlorogenin, Eruboside, Protoeruboside, Manogenin, Shlorogenin, Hainangenin, Protodioscin, Protodiosgenin, Aculeoside, Smilagenin, Sarsapogenin, Yamogenin, Yuccagenin, Gracillin, Sativoside, and combinations thereof.

15. The method according to claim 14, wherein such a plant extract that contains a saponin or sapogenin is present in a composition, and represents from 0.01 to 20.0% of the total weight of the composition, the composition further comprising a physiologically acceptable medium.

16. A method for the dermatological treatment of disorders associated with excess sebum production, comprising applying to skin exhibiting a disorder associated with excess sebum production a furostanol saponin and/or sapogenin.

17. The method according to claim 16, wherein such saponin or sapogenin is present in a composition, and represents from 0.01 to 5.0% of the total weight of the composition, the composition further comprising a physiologically acceptable medium.

18. The method according to claim 16, comprising applying a plant extract that contains a saponin or sapogenin selected from the group consisting of Dioscin, Diosgenin, Hecogenin, Heconin, Tigogenin, Tigonin, Gitogenin, Chlorogenin, Eruboside, Protoeruboside, Manogenin, Shlorogenin, Hainangenin, Protodioscin, Protodiosgenin, Aculeoside, Smilagenin, Sarsapogenin, Yamogenin, Yuccagenin, Gracillin, Sativoside, and combinations thereof.

19. The method according to claim 18, wherein such a plant extract that contains a saponin or sapogenin is present in a composition, and represents from 0.01 to 20.0% of the total weight of the composition, the composition further comprising a physiologically acceptable medium.
